(19) United States
(12) Patent Application Publication
(10) Pub. No.: US 2018/0049713 A1
(43) Pub. Date: Feb. 22, 2018

(54) CONTROL APPARATUS, CONTROL SYSTEM, PROCESSING METHOD, AND NON-TRANSITORY COMPUTER-READABLE STORAGE MEDIUM

(71) Applicant: CANON KABUSHIKI KAISHA, Tokyo (JP)

(72) Inventor: Daiya Semba, Inagi-shi (JP)

(21) Appl. No.: 15/673,493

(22) Filed: Aug. 10, 2017

(30) Foreign Application Priority Data
Aug. 16, 2016 (JP) 2016-159680

(51) Int. Cl. A61B 6/00; G06F 19/00

(52) U.S. Cl. CPC A61B 6/5294; G06F 19/321

(57) ABSTRACT

A control apparatus comprises an instruction unit configured to receive an instruction to change one of a captured image and an image that is a processed image based on the captured image to a rejected image; and an assigning unit configured to assign information representing an imaging success/failure to the image based on information of an imaging protocol of the image and information representing whether the image is the rejected image instructed by the instruction unit.

### BACKGROUND OF THE INVENTION

### Field of the Invention

The present invention relates to a control apparatus, a control system, and a processing method.

### Description of the Related Art

In a medical institution, a medical information system by network connection is constructed. For example, if it is determined that radiation imaging is necessary, an examination instruction is input from an HIS (Hospital Information System) terminal or an RIS (Radiology Information System) terminal and transmitted to a radiography department that is the request destination. These network communications are implemented by a technical framework based on a medical standard specification guideline called IHE (Integrating the Healthcare Enterprise).

Request information transmitted by the HIS or RIS is called an examination order. The examination order includes a request source department name, an examination item, the personal data of a patient, and the like. When the radiography department receives the examination order, an imaging protocol necessary for imaging is input to a radiation imaging system. Then, the examination progresses based on the examination order including a plurality of imaging protocols. Here, an imaging protocol includes an imaging part and an imaging method requested by a doctor, information of image processing, and the like. An image obtained by imaging is stored in association with imaging information such as an imaging date/time and an exposure dose. During the examination, a captured image unsuitable for a diagnosis is sometimes obtained due to a failure of an imaging technique by an imaging technician. In this case, a rejected image instruction is input to the radiation imaging system, thereby storing the captured image as a rejected image. At the end of the examination, captured images are transferred to a medical image management system called a PACS (Picture Archiving and Communication Systems) and saved.

Japanese Patent Laid-Open No. 2001-175771 discloses a technique of controlling to transfer only effective images to the PACS and not to transfer rejected images to the PACS because transfer of the rejected images to the PACS increases the network load and causes a squeeze on the capacity of the PACS.

Japanese Patent Laid-Open No. 2007-44272 discloses a technique of automatically acquiring and managing rejected images and the imaging information of the rejected images, for the purpose of improving the skill of an imaging technician and reducing ineffective exposure of a patient along with the skill improvement. This makes it possible to transfer all pieces of information about rejected images to a rejected image statistic server.

In the medical field, the imaging technician may generate a number of images until images suitable for a diagnosis, for example, a projected image obtained by tomosynthesis imaging and a reconstructed image based on the projected image, or a captured image and a replicated image based on the captured image are obtained.

However, images that are handled as rejected images not to transfer images other than images suitable for a diagnosis to the PACS may include images that are not images of an imaging failure. The images handled as rejected images are transferred to the rejected image statistic server. In this case, even the images that are not images of an imaging failure and are originally not necessary for rejected image analysis are transferred to the rejected image statistic server. This requires time to select images necessary for rejected image analysis and lowers the efficiency of rejected image analysis.

The present invention has been made in consideration of the above-described problems, and provides a technique of reducing the time to select images necessary for rejected image analysis and improving the efficiency of rejected image analysis.

### SUMMARY OF THE INVENTION

According to one aspect of the present invention, there is provided a control apparatus comprising: an instruction unit configured to receive an instruction to change one of a captured image and an image that is a processed image based on the captured image to a rejected image; and an assigning unit configured to assign information representing an imaging success/failure to the image based on information of an imaging protocol of the image and information representing whether the image is the rejected image instructed by the instruction unit.

According to one aspect of the present invention, there is provided a control apparatus comprising: an instruction unit configured to receive an instruction to change an image to a rejected image; a determination unit configured to determine whether to output the rejected image to a rejected image statistic terminal; and an output unit configured to output the rejected image determined, by the determination unit, to be output to the rejected image statistic terminal.

According to one aspect of the present invention, there is provided a control system comprising: an instruction unit configured to receive an instruction to change one of a captured image and an image that is a processed image based on the captured image to a rejected image; and an assigning unit configured to assign information representing an imaging success/failure to the image based on information of an imaging protocol of the image and information representing whether the image is the rejected image instructed by the instruction unit.

According to one aspect of the present invention, there is provided a processing method executed by a control apparatus, comprising: receiving an instruction to change one of a captured image and an image that is a processed image based on the captured image to a rejected image; and assigning information representing an imaging success/failure to the image based on information of an imaging protocol of the image and information representing whether the image is the rejected image instructed in the receiving the instruction.

According to one aspect of the present invention, there is provided a processing method executed by a control apparatus, comprising: receiving an instruction to change an image to a rejected image; determining whether to output the rejected image to a rejected image statistic terminal; and outputting the rejected image determined, in the determining whether to output the rejected image, to be output to the rejected image statistic terminal.

Further features of the present invention will become apparent from the following description of exemplary embodiments with reference to the attached drawings.

### BRIEF DESCRIPTION OF THE DRAWINGS

FIG. 1 is a block diagram showing an example of the arrangement of a control system according to the first to fourth embodiments;

FIG. 2 is a block diagram showing an example of the functional arrangement of an X-ray imaging control unit according to the first and third embodiments;

FIG. 3 is a view showing an example of an imaging screen according to the first, third, and fifth embodiments;

FIG. 4 is a flowchart showing an example of the imaging operation of an X-ray imaging system according to the first to sixth embodiments;

FIG. 5 is a flowchart showing an example of imaging processing and post-processing according to the first, third, and fifth embodiments;

FIG. 6 is a table showing an example of examination information according to the first, third, and fifth embodiments;

FIG. 7 is a flowchart showing an example of the operation of an imaging success/failure identification unit according to the first, third, and fifth embodiments;

FIG. 8 is a flowchart showing an example of the operation of a rejected image statistic output unit according to the first and second embodiments;

FIG. 9 is a block diagram showing an example of the functional arrangement of an X-ray imaging control unit according to the second and fourth embodiments;

FIG. 10 is a view showing an example of an imaging screen according to the second, fourth, and sixth embodiments;

FIG. 11 is a flowchart showing an example of imaging processing and post-processing according to the second, fourth, and sixth embodiments;

FIG. 12 is a table showing an example of examination information according to the second, fourth, and sixth embodiments;

FIG. 13 is a flowchart showing an example of the operation of an imaging success/failure identification unit according to the second, fourth, and sixth embodiments;

FIG. 14 is a flowchart showing an example of the operation of a rejected image statistic output unit according to the third to sixth embodiments;

FIG. 15 is a block diagram showing an example of the arrangement of a control system according to the fifth and sixth embodiments;

FIG. 16 is a block diagram showing an example of the functional arrangement of an X-ray imaging control unit according to the fifth embodiment; and

FIG. 17 is a block diagram showing an example of the functional arrangement of an X-ray imaging control unit according to the sixth embodiment.

### DESCRIPTION OF THE EMBODIMENTS

An exemplary embodiment(s) of the present invention will now be described in detail with reference to the drawings. It should be noted that the relative arrangement of the components, the numerical expressions and numerical values set forth in these embodiments do not limit the scope of the present invention unless it is specifically stated otherwise.

In the embodiments to be described below, an example in which X-rays are applied as radiation will be described. However, the radiation is not limited to X-rays and may be, for example, an electromagnetic wave, α-rays, β-rays, or γ-rays. Note that arrangements to be described in the following embodiments are merely examples, and the present invention is not limited to the illustrated arrangements.

### First Embodiment

<Outline>

In the first embodiment, an example will be described in which an instruction to handle a captured image or an image that is a processed image based on the captured image as a rejected image is received, and information representing an imaging success/failure is assigned to the image based on the information of the imaging protocol of the image and information representing whether the image is the instructed rejected image. More specifically, if a projected image captured using a tomosynthesis imaging protocol and reconstructed images generated from the projected image include an effective image (an image that is not rejected image-instructed), information representing an imaging success is assigned to all images.

If the projected image and the reconstructed images include an effective image, it is considered that images other than the specific effective image should not be output to a rejected image statistic terminal (rejected image statistic server), and therefore, a rejected image instruction is done for the other images (although they are not images of an imaging failure). For this reason, if an effective image is included, information representing an imaging success is assigned to all images. This can prevent an image that is rejected image-instructed but is not an image of an imaging failure from being output to the rejected image statistic terminal.

On the other hand, if no effective image is included, information representing an imaging failure is assigned to all images. Then, each image with the assigned information representing an imaging failure and the additional information (including at least one piece of information of a protocol number, an imaging protocol, a re-imaging source protocol number, an image number, an image type, information representing a rejected image state, and information representing an imaging success/failure) of the image are output to the rejected image statistic terminal. An image that has failed in imaging can thus be output to the rejected image statistic terminal.

In this way, when the information representing the imaging success/failure is assigned separately from the information representing the rejected image state, an image that has failed in imaging and is needed for rejected image analysis can be output. Hence, the efficiency of rejected image analysis can be improved. The first embodiment will be described below in detail.

<Arrangement of Control System>

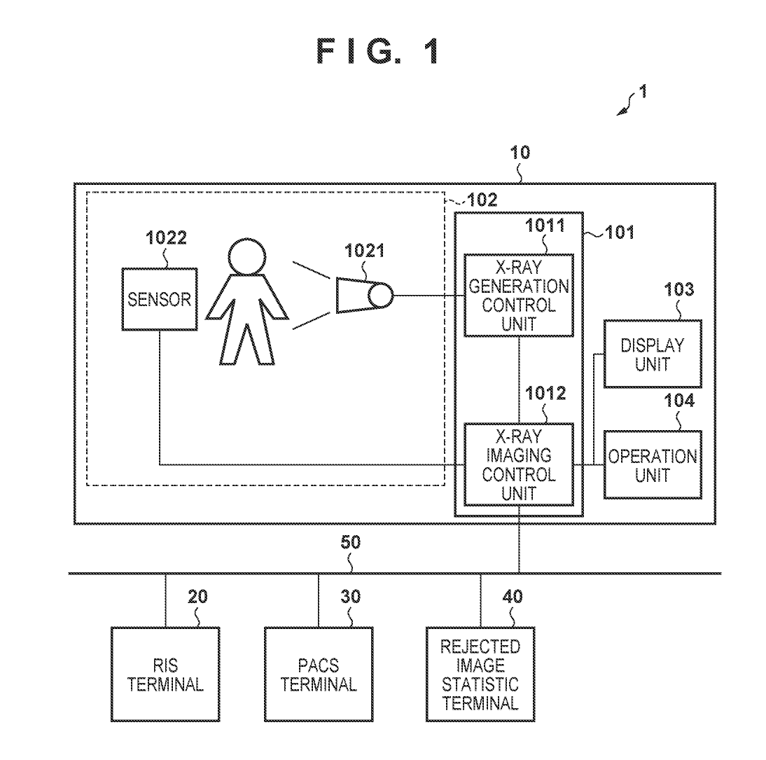

FIG. 1 is a block diagram showing an example of the arrangement of a control system according to an embodiment. A control system 1 includes an X-ray imaging system 10, an RIS terminal 20, a PACS terminal 30, and a rejected image statistic terminal 40. These devices are connected via, for example, a communication unit 50 such as a network.

The X-ray imaging system 10 captures an X-ray digital image (to be referred to as a captured image hereinafter). The RIS terminal 20 is an information system in a radiography department. The PACS terminal 30 saves and manages the image captured by the X-ray imaging system 10. The rejected image statistic terminal 40 saves and manages images that have failed in imaging. Note that the rejected image statistic terminal 40 need not always be separated from the X-ray imaging system 10, as shown in FIG. 1, and may exist in the X-ray imaging system 10. The rejected image statistic terminal 40 may be provided in a control apparatus 101 to be described later. Alternatively, the rejected image statistic terminal 40 may be integrated with the RIS terminal 20 and/or the PACS terminal 30.

The X-ray imaging system 10 executes an examination (imaging) based on an examination order formed from a plurality of imaging protocols. Each of the imaging protocols defines imaging conditions, the contents of image processing to be executed for a captured image, and the like. More specifically, each imaging protocol includes various kinds of parameter information such as an imaging time and image processing and imaging execution information. In addition, imaging environment information (a sensor type and an imaging posture) is associated with the imaging protocol.

<Arrangement of X-Ray Imaging System>

The X-ray imaging system 10 includes the control apparatus 101, an imaging unit 102, a display unit 103, and an operation unit 104.

The control apparatus 101 includes an X-ray generation control unit 1011 and an X-ray imaging control unit 1012. The X-ray generation control unit 1011 controls generation of X-rays from an X-ray tube 1021. More specifically, the X-ray generation control unit 1011 applies a voltage to the X-ray tube 1021 based on imaging conditions (for example, parameters such as a tube current, a tube voltage, and an irradiation time) corresponding to an imaging protocol to cause the X-ray tube 1021 to generate X-rays. The X-ray imaging control unit 1012 generally controls X-ray imaging processing by a sensor 1022 based on the imaging protocol. The X-ray imaging control unit 1012 also performs, for example, image processing (for example, correction processing, tone processing, or frequency processing) for a captured image. The image processing is performed using image processing parameters corresponding to the imaging protocol at the time of imaging.

The imaging unit 102 includes the X-ray tube 1021 and the sensor 1022. The X-ray tube 1021 functions as a radiation generator and irradiates an object (that is, a subject) with X-rays. The sensor 1022 is an FPD (Flat Panel Detector) functioning as a radiation detector and detects the X-rays transmitted through the subject. The sensor 1022 A/D-converts charges corresponding to the detected amount of X-rays transmitted through the subject and transfers the data to the X-ray imaging control unit 1012 as a captured image.

The display unit 103 is formed from, for example, a display and displays a system state and the like to the operator. The display unit 103 displays, for example, an examination order received from the RIS terminal 20 (or an examination order created by the operator). The operation unit 104 is formed from, for example, a keyboard, a mouse, and various kinds of buttons, and inputs an instruction from the operator to the apparatus. The operator inputs, for example, an image replication instruction by operating the operation unit 104.

<Arrangement of X-Ray Imaging Control Unit>

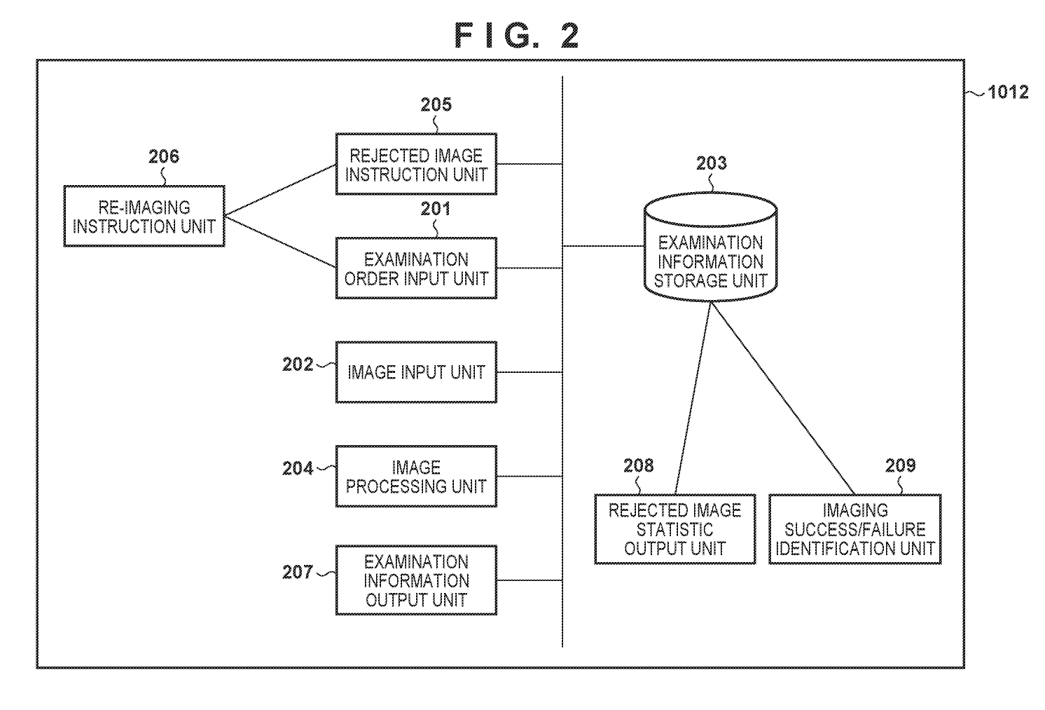

FIG. 2 is a block diagram showing an example of the arrangement of the X-ray imaging control unit according to an embodiment. The X-ray imaging control unit 1012 includes an examination order input unit 201, an image input unit 202, an examination information storage unit 203, an image processing unit 204, and a rejected image instruction unit 205. The X-ray imaging control unit 1012 also includes a re-imaging instruction unit 206, an examination information output unit 207, a rejected image statistic output unit 208, and an imaging success/failure identification unit 209.

The examination order input unit 201 inputs an examination order. The examination order is examination information including the information of a patient to be captured and imaging contents. The examination order is acquired from an external system such as the RIS terminal 20 or input via an operation on the operation unit 104. The image input unit 202 inputs a radiation image captured by the X-ray imaging system 10. The radiation image is input via a network or input via a medium such as a CD-ROM or a DVD in some cases.

The examination information storage unit 203 stores the image input from the image input unit 202 and the examination information input from the examination order input unit 201 in association with each other. The image processing unit 204 performs image processing such as correction processing, tone processing, or frequency processing for the captured image using image processing parameters corresponding to the imaging protocol.

The rejected image instruction unit 205 assigns information representing a rejected image state to the captured image as additional information, and changes the captured image to a rejected image. The rejected image instruction unit 205 stores the change contents in the examination information storage unit 203. The re-imaging instruction unit 206 prepares for re-imaging concerning an executed imaging protocol. More specifically, the re-imaging instruction unit 206 changes a captured image of a designated executed imaging protocol to a rejected image by controlling the rejected image instruction unit 205. Additionally, the re-imaging instruction unit 206 adds the same imaging protocol as the executed imaging protocol to the examination by controlling the examination order input unit 201 such that the same imaging can be performed.

The examination information output unit 207 outputs examination information stored in the examination information storage unit 203 to the PACS terminal 30. The rejected image statistic output unit 208 outputs, of the information stored in the examination information storage unit 203, a captured image and additional information thereof to the rejected image statistic terminal 40. The imaging success/failure identification unit 209 identifies, based on the examination information and the additional information of the captured image stored in the examination information storage unit 203, whether the imaging has succeeded or failed concerning the captured image.

<Example of Imaging Screen>

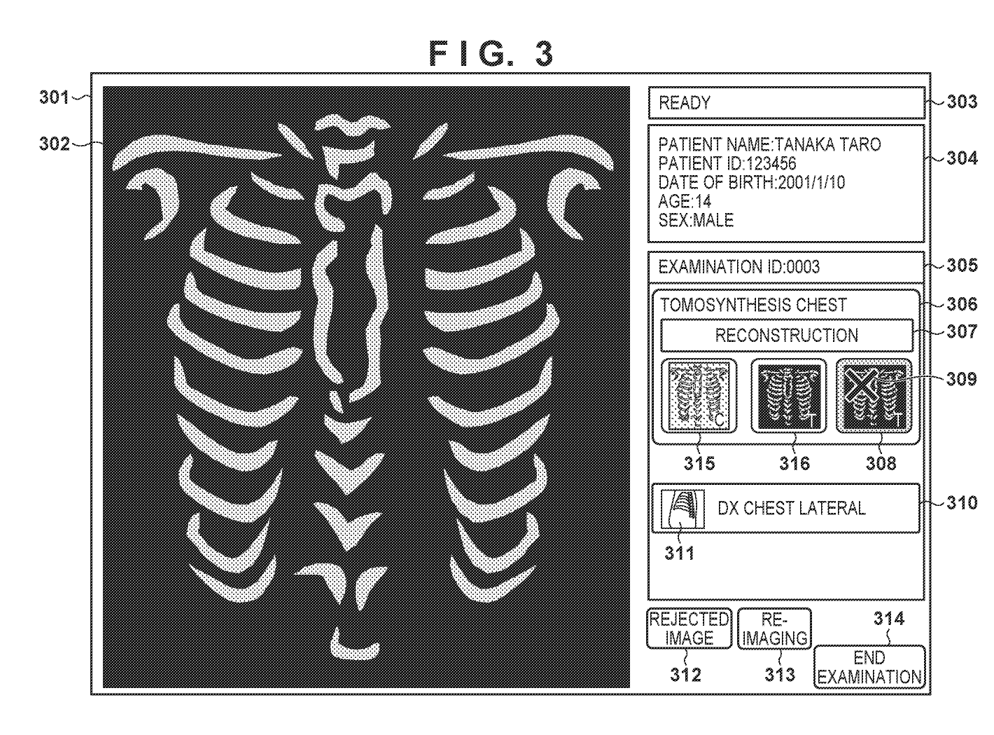

FIG. 3 shows an example of an imaging screen according to an embodiment of the present invention. An imaging screen 301 includes a preview image display region 302 that displays a preview of a captured image, and a sensor state display region 303 that displays the state of the sensor 1022 concerning an imaging protocol under execution. The imaging screen 301 also includes a patient information display region 304, an examination order display region 305, a rejected image button 312 that instructs change of a captured image to a rejected image state, a re-imaging button 313 that instructs re-imaging, and an examination end button 314 that instructs termination of examination.

The examination order display region 305 displays imaging protocols 306 and 310 included in an examination. The name of an imaging technique is displayed in correspondence with each imaging protocol. The imaging protocols are provided with thumbnail display regions 308, 311, 315, and 316 that display the thumbnails of images captured and generated by the imaging protocols. A symbol 309 representing a rejected image is displayed on the thumbnail of a rejected image. The imaging protocol 306 is a tomosynthesis imaging protocol, and a reconstruction button 307 that instructs reconstructed image generation is displayed.

<Processing>

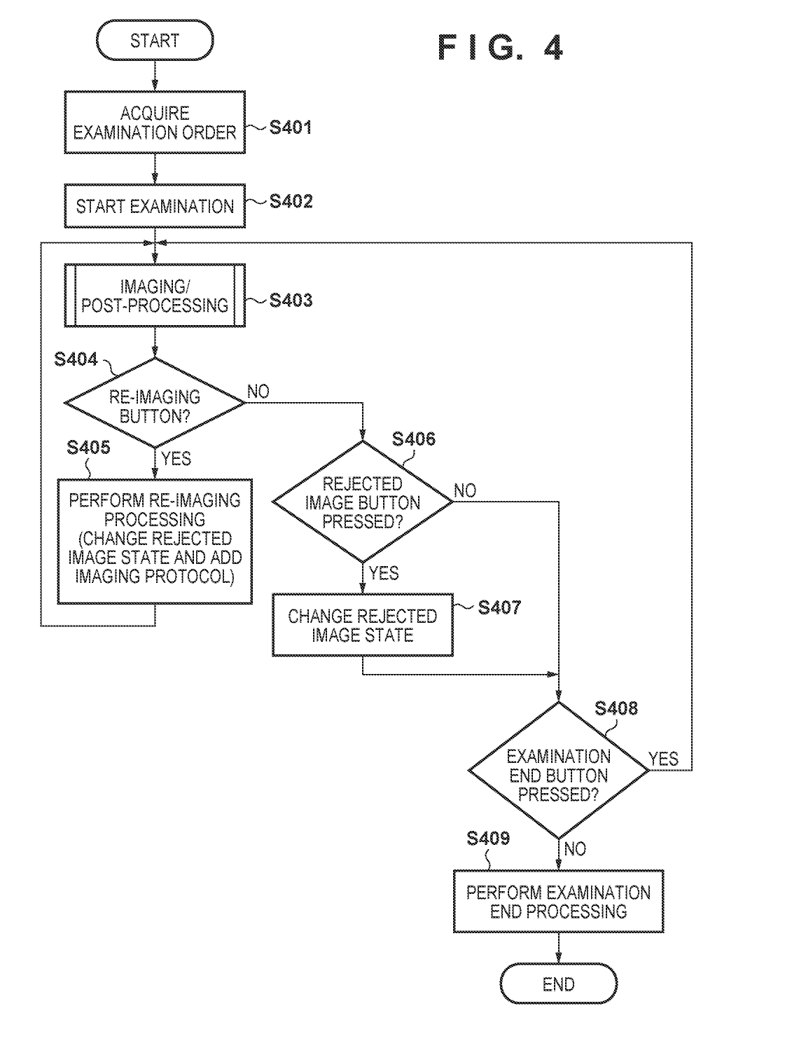

The procedure of a series of processes from the start to the end of an examination executed by the control apparatus 101 according to an embodiment of the present invention will be described next with reference to the flowchart of FIG. 4.

In step S401, the control apparatus 101 acquires an examination order from the RIS terminal 20 (or an examination order manually created by the operator) via the examination order input unit 201, and stores examination information included in the acquired examination order in the examination information storage unit 203.

In step S402, the control apparatus 101 starts an examination and displays the imaging screen 301 on the display unit 103.

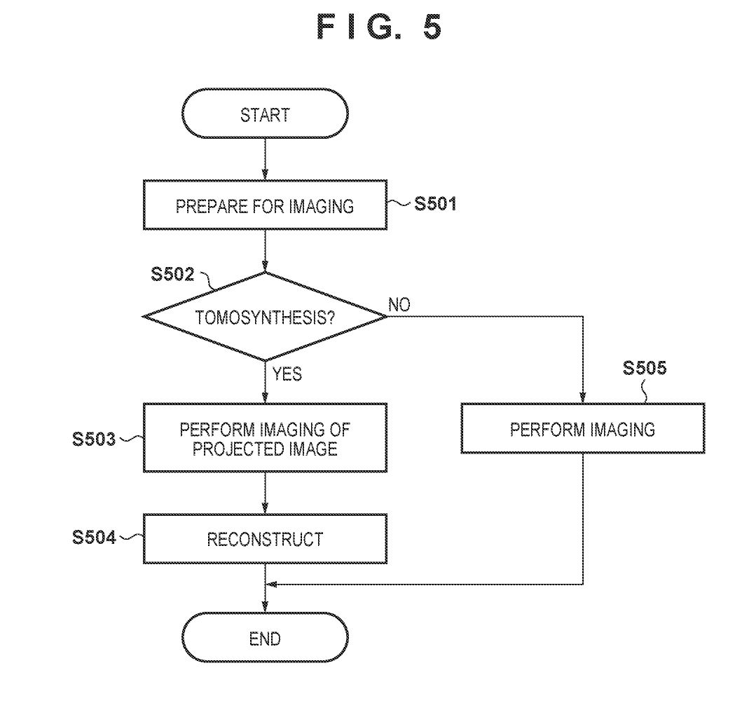

In step S403, the control apparatus 101 executes imaging processing and post-processing for the captured image. Details of the processing of step S403 will be described here with reference to the flowchart of FIG. 5.

In step S501, the control apparatus 101 makes preparations for imaging. More specifically, the control apparatus 101 changes an unexecuted imaging protocol to an imaging preparation state in accordance with a predetermined order (for example, sequentially from the top of the examination order), and also makes preparations for imaging by controlling the X-ray tube 1021 and the sensor 1022 based on imaging conditions included in the unexecuted imaging protocol. The X-ray tube 1021 is controlled by the X-ray generation control unit 1011, and the sensor 1022 is controlled by the X-ray imaging control unit 1012.

In step S502, the control apparatus 101 determines whether the imaging protocol is a tomosynthesis imaging protocol. If the imaging protocol is a tomosynthesis imaging protocol (YES in step S502), the process advances to step S503. On the other hand, if the imaging protocol is not a tomosynthesis imaging protocol (NO in step S502), the process advances to step S505.

In step S503, the control apparatus 101 captures a projected image serving as the source of a reconstructed image later. More specifically, the X-ray tube 1021 irradiates the object with X-rays, and the sensor 1022 detects the X-rays transmitted through the object. The image input unit 202 acquires a projected image obtained as a result, and the image processing unit 204 performs image processing such as correction processing, tone processing, or frequency processing for the acquired projected image. The projected image is associated with the examination information and stored in the examination information storage unit 203. At this time, the imaging screen 301 displays the projected image and additional image information. The projected image is displayed in the preview image display region 302, and the thumbnail of the projected image is displayed in the thumbnail display region 315 on the tomosynthesis imaging protocol used for the imaging.

In step S504, according to the pressing of the reconstruction button 307 by the operator via an operation on the operation unit 104, the control apparatus 101 causes the image processing unit 204 to reconstruct the projected image to generate a tomographic image (reconstructed image). A reconstructed image can be generated not only once but a plurality of times. For example, the operator sometimes generates several reconstructed images while adjusting reconstruction parameters via an operation on the operation unit 104 so as to obtain a reconstructed image suitable for a diagnosis. The thumbnails of the generated reconstructed images are displayed in the thumbnail display regions 316 and 308. After that, the process advances to step S404.

In step S505, the control apparatus 101 performs imaging using a protocol other than the tomosynthesis imaging protocol, for example, a still imaging protocol. More specifically, the X-ray tube 1021 irradiates the object with X-rays, and the sensor 1022 detects the X-rays transmitted through the object. The image input unit 202 acquires a captured image obtained as a result, and the image processing unit 204 performs image processing such as correction processing, tone processing, or frequency processing for the acquired captured image. The captured image is associated with the examination information and stored in the examination information storage unit 203. At this time, the imaging screen 301 displays the captured image and additional image information. The captured image is displayed in the preview image display region 302, and the thumbnail of the captured image is displayed in the thumbnail display region 311 on the imaging protocol 310 used for the imaging. After that, the process advances to step S404.

Referring back to the flowchart of FIG. 4, in step S404, the control apparatus 101 determines whether the operator presses the re-imaging button 313 via an operation on the operation unit 104. For example, if the operator views the image displayed in the preview image display region 302 and determines that the image is inappropriate for a diagnosis, and re-imaging is necessary, the re-imaging button 313 is pressed. If the re-imaging button 313 is pressed (YES in step S404), the process advances to step S405. On the other hand, if the re-imaging button 313 is not pressed (NO in step S404), the process advances to step S406.

In step S405, the control apparatus 101 performs re-imaging processing. More specifically, the control apparatus 101 instructs various kinds of operations via the re-imaging instruction unit 206. First, the rejected image instruction unit 205 changes a captured image designated when the re-imaging button 313 is pressed in step S404 to a rejected image state under the control of the re-imaging instruction unit 206. Next, the examination order input unit 201 adds the same imaging protocol as the imaging protocol used to obtain the captured image in the rejected image state to the examination under the control of the re-imaging instruction unit 206. The re-imaging instruction unit 206 stores the contents of the change by the rejected image instruction unit 205 and the examination order input unit 201 in the examination information storage unit 203. After that, the process returns to step S403.

In step S406, the control apparatus 101 determines whether the rejected image button 312 is pressed via an operation on the operation unit 104. For example, assume that the operator views the captured image displayed in the preview image display region 302 and determines that the captured image is inappropriate for a diagnosis. Alternatively, assume that the operator selects one of a plurality of reconstructed images of tomosynthesis imaging as an image appropriate for a diagnosis, as shown in, for example, the thumbnail display regions 316 and 308, and determines that the remaining images should be changed to a rejected image state not to transfer them to the PACS terminal 30. As a result, the rejected image button 312 is pressed via an operation on the operation unit 104. If the rejected image button 312 is pressed (YES in step S406), the process advances to step S407. On the other hand, if the rejected image button 312 is not pressed (NO in step S406), the process advances to step S408.

In step S407, the control apparatus 101 causes the rejected image instruction unit 205 to change the designated captured image to the rejected image state, and stores the change contents in the examination information storage unit 203. After that, the process advances to step S408.

In step S408, the control apparatus 101 determines whether the examination end button 314 is pressed via an operation on the operation unit 104. For example, the operator determines whether the imaging processes of all imaging protocols are completed by confirming the examination order display region 305 displayed on the display unit 103. If the operator determines that the imaging processes of all imaging protocols are ended or wants to end the examination, he/she presses the examination end button 314 via the operation unit 104. If an unexecuted imaging protocol remains, and the examination should be continued, the operator does not press the examination end button 314. If the examination end button 314 is pressed (YES in step S408), the process advances to step S409. On the other hand, if the examination end button 314 is not pressed (NO in step S408), the process returns to step S403.

In step S409, the control apparatus 101 performs examination end processing. More specifically, the examination information output unit 207 outputs, of the information stored in the examination information storage unit 203, an effective image that is not in the rejected image state and the additional information of the effective image to the PACS terminal 30, thereby ending the processing. The series of processes of the flowchart in FIG. 4 thus ends.

<Example of Examination Information>

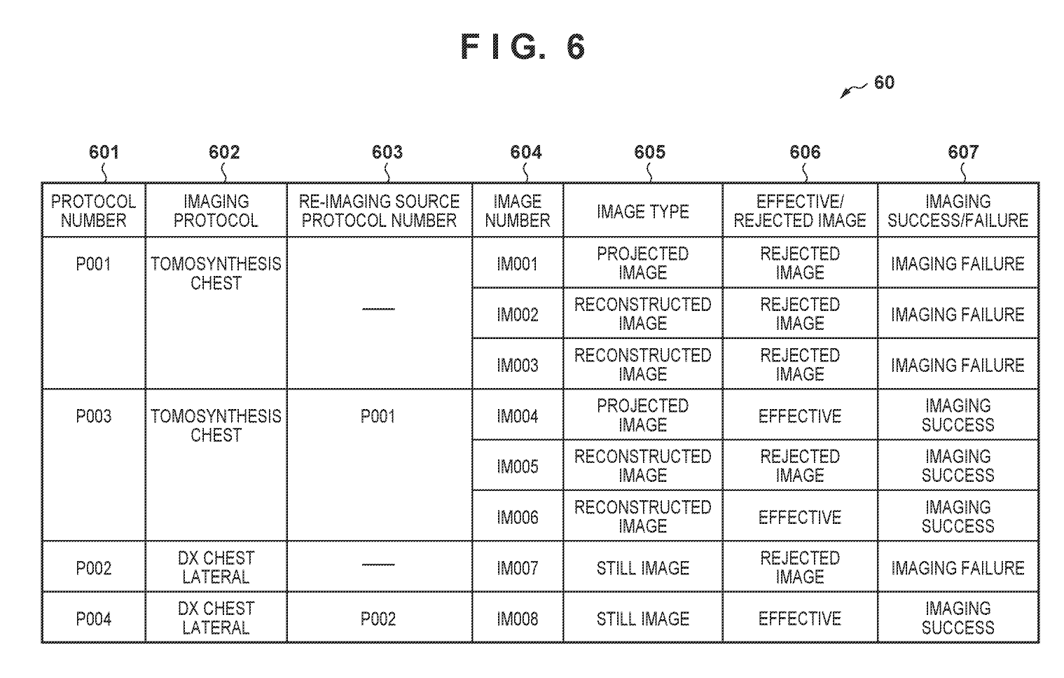

An example of examination information according to an embodiment of the present invention will be described next with reference to FIG. 6. A table 60 shows some pieces of examination information after the end of an examination, which are stored in the examination information storage unit 203, in a table format.

A protocol number item 601 represents a number assigned to an imaging protocol. The number is incremented like P001, P002, . . . in the order of addition to an examination order. An imaging protocol item 602 represents the name of an imaging protocol. The imaging protocol is a tomosynthesis imaging protocol or DX imaging protocol. An imaging technique or an imaging part is used as a name in the imaging protocol item 602, like “tomosynthesis chest” or “DX chest lateral”.

A re-imaging source protocol number item 603 is the number of the protocol number item of the re-imaging source assigned to a re-imaging protocol added by the processing of the re-imaging instruction unit 206. For example, the imaging protocol of protocol number P003 in the table 60 is an imaging protocol added for re-imaging of imaging protocol P001. The re-imaging source protocol number item 603 is blank for an imaging protocol that is not added in re-imaging processing.

An image number item 604 represents a number assigned to an image. The number is incremented like IM001, IM002, . . . in the order of obtaining by imaging or generation. An image type item 605 represents an image type such as a projected image, a reconstructed image, or a still image. An effective/rejected image item 606 represents whether an image is effective or in a rejected image state.

This item is a “rejected image” for images changed to the rejected image state by the rejected image instruction unit 205. This item is “effective” for the remaining images. An imaging success/failure item 607 represents whether an imaging technique has succeeded or failed, and success/failure information identified by the imaging success/failure identification unit 209 is assigned.

<Acquisition Processing of Items (Other than Imaging Success/Failure Item 607) of Table 60>

What kind of processing is performed up to the end of an examination to obtain the pieces of examination information other than the imaging success/failure item 607 of the table 60 shown in FIG. 6 will be described below with reference to FIGS. 4 and 5 again. First, imaging protocols P001 and P002 are input in step S401 of FIG. 4, and the examination is started in step S402. Imaging is performed first based on tomosynthesis imaging protocol P001. For this reason, in the imaging processing and post-processing of step S403, the process advances to step S503 in FIG. 5. Projected image IM001 is thus acquired.

Next, assume that reconstructed images IM002 and IM003 are acquired based on projected image IM001 in step S504. Note that reconstructed images IM002 and IM003 undergo image processing using different reconstruction parameters. If the operator observes reconstructed images IM002 and IM003 and understands that the imaging technique for projected image IM001 has failed, he/she presses the re-imaging button 313 in step S404.

In step S405, all images IM001, IM002, and IM003 obtained by imaging protocol P001 are changed to the rejected image state. Imaging protocol P003 that is the same as imaging protocol P001 is added to the examination order. After that, the process returns to step S403. To further perform imaging based on re-imaging protocol P003, the process similarly advances to step S503 of FIG. 5, and projected image IM004 is obtained.

Next, in step S504, reconstructed images IM005 and IM006 are acquired based on projected image IM004. Note that reconstructed images IM005 and IM006 undergo image processing using different reconstruction parameters. Assume that the operator compares reconstructed images IM005 and IM006 and determines that reconstructed image IM006 is more appropriate for a diagnosis.

Here, since effective image IM006 is included, the re-imaging button 313 is not pressed in step S404, and the process advances to step S406. In step S406, to prevent reconstructed image IM005 inappropriate for a diagnosis from being transferred to the PACS terminal 30, the rejected image button 312 is pressed. As a result, in step S407, reconstructed image IM005 is changed to the rejected image state.

In step S408, since unexecuted imaging protocol P002 remains, the examination end button 314 is not pressed, and the process returns to step S403. Since the imaging protocol P002 is an imaging protocol other than a tomosynthesis imaging protocol, the process advances to step S505 as a result of determination processing of step S502. Still image IM007 is obtained by imaging in step S505. Assume that the operator observes still image IM007, understands that the imaging technique has failed, and presses the re-imaging button 313 in step S404.

In step S405, image IM007 obtained by imaging protocol P002 is changed to the rejected image state. Imaging protocol P004 that is the same as imaging protocol P002 is added to the examination order. After that, the process returns to step S403. To further perform imaging using re-imaging protocol P002, the process similarly advances to step S505, and still image IM008 is obtained. The operator is assumed to determine that still image IM008 is appropriate for a diagnosis.

Hence, the re-imaging button 313 is not pressed in step S404, the rejected image button 312 is not pressed in step S406, and the process advances to step S408. Here, since the imaging processes of all imaging protocols are completed, the examination end button 314 is pressed in step S408, and the examination ends. By the series of processes described above, the items of the table 60 except the imaging success/failure item in FIG. 6 are obtained.

<Acquisition Processing of Imaging Success/Failure Item 607 of Table 60>

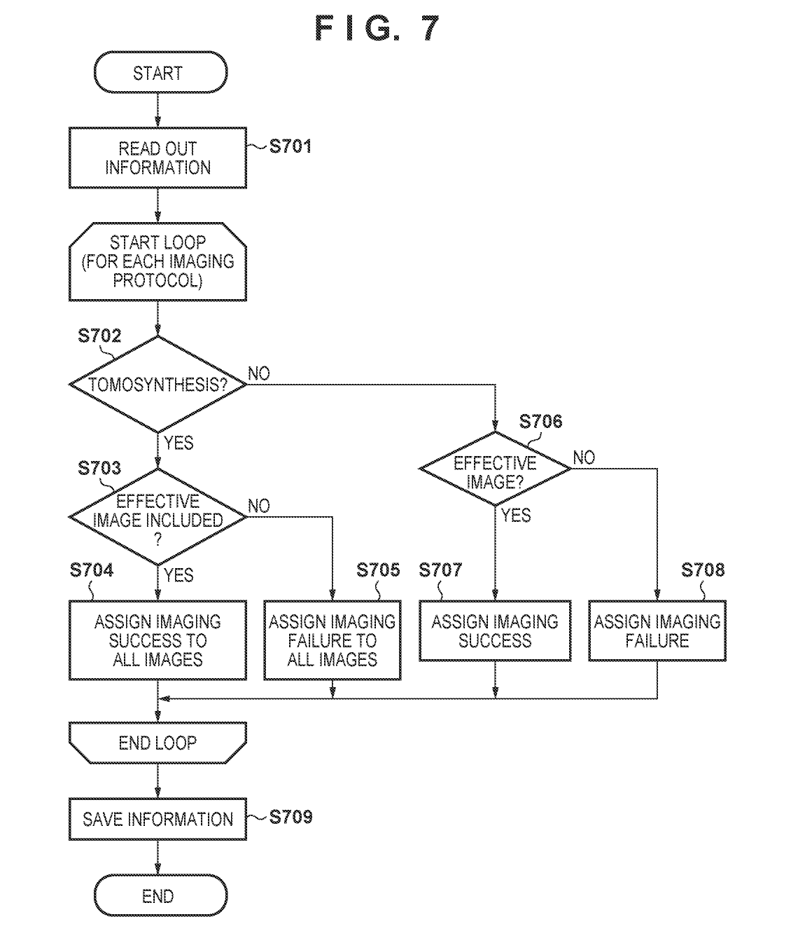

The procedure of processing executed by the imaging success/failure identification unit 209 for examination information after the end of the examination according to an embodiment of the present invention will be described next with reference to the flowchart of FIG. 7. What kind of processing is performed to obtain the imaging success/failure item 607 of the table 60 shown in FIG. 6 will be described here. Note that the processing of the imaging success/failure identification unit 209 is executed at an arbitrary timing after the end of the examination until the rejected image statistic output unit 208 outputs information to the rejected image statistic terminal 40. For example, the processing is executed at the end of examination, at the timing of an execution instruction via the operation unit 104, or at the timing of information output to the rejected image statistic terminal 40.

In step S701, the imaging success/failure identification unit 209 reads out examination information to be processed from the examination information storage unit 203. Here, the examination information of the table 60 shown in FIG. 6 is read out. Next, the processes of steps S702 to S708 are performed as a loop for the imaging protocols in the examination information sequentially from the top. After the end of the loop, the process advances to step S709. First, processing is performed for imaging protocol P001 at the top in the table 60.

In step S702, the imaging success/failure identification unit 209 determines whether the imaging protocol is a tomosynthesis imaging protocol. If the imaging protocol is a tomosynthesis imaging protocol (YES in step S702), the process advances to step S703. On the other hand, if the imaging protocol is another imaging protocol (NO in step S702), the process advances to step S706. Since imaging protocol P001 is a tomosynthesis imaging protocol, the process advances to step S703 here.

In step S703, the imaging success/failure identification unit 209 determines whether an effective image is included in the projected image and all reconstructed images acquired by the tomosynthesis imaging protocol. Upon determining that an effective image is included (YES in step S703), the process advances to step S704. On the other hand, upon determining that no effective image is included (NO in step S703), the process advances to step S705. Here, since all images IM001, IM002, and IM003 acquired by imaging protocol P001 are rejected images, the process advances to step S705.

In step S705, the imaging success/failure identification unit 209 assigns imaging failure information to the projected image and all reconstructed images acquired by the tomosynthesis imaging protocol. Here, imaging failure information is assigned to images IM001, IM002, and IM003. After that, the process returns to step S702 at the start of the loop, and processing is performed for imaging protocol P003 next.

Since imaging protocol P003 is a tomosynthesis imaging protocol (YES in step S702), the imaging success/failure identification unit 209 advances to step S703. Since imaging protocol P003 includes effective images IM004 and IM006 (YES in step S703), the process advances to step S704.

In step S704, the imaging success/failure identification unit 209 assigns imaging success information to the projected image and all reconstructed images acquired by the tomosynthesis imaging protocol. Here, imaging success information is assigned to images IM004, IM005, and IM006. After that, the process returns to step S702 at the start of the loop, and processing is performed for imaging protocol P002 next.

Since imaging protocol P002 is not a tomosynthesis imaging protocol (NO in step S702), the process advances to step S706. In step S706, the imaging success/failure identification unit 209 determines whether the image captured by the imaging protocol is an effective image. If the image is an effective image (YES in step S706), the process advances to step S707. On the other hand, if the image is not an effective image (NO in step S706), the process advances to step S708. Here, since image IM007 acquired by imaging protocol P002 is a rejected image, the process advances to step S708.

In step S708, the imaging success/failure identification unit 209 assigns imaging failure information to the image captured by the imaging protocol. Here, imaging failure information is assigned to image IM007. After that, the process returns to step S702 at the start of the loop, and processing is performed for imaging protocol P004 next.

Since imaging protocol P004 is not a tomosynthesis imaging protocol (NO in step S702), the process advances to step S706. Since image IM008 acquired by imaging protocol P004 is an effective image (YES in step S706), the process advances to step S707.

In step S707, the imaging success/failure identification unit 209 assigns imaging success information to the image captured by the imaging protocol. Here, imaging success information is assigned to image IM008. Since the loop processing is thus completed for all imaging protocols, the process advances to step S709.

In step S709, the imaging success/failure identification unit 209 stores the examination information changed by the series of processes of steps S702 to S708 in the examination information storage unit 203. By the processes described above, the information of the imaging success/failure item 607 of the table 60 is obtained.

<Processing of Rejected Image Statistic Output Unit 208>

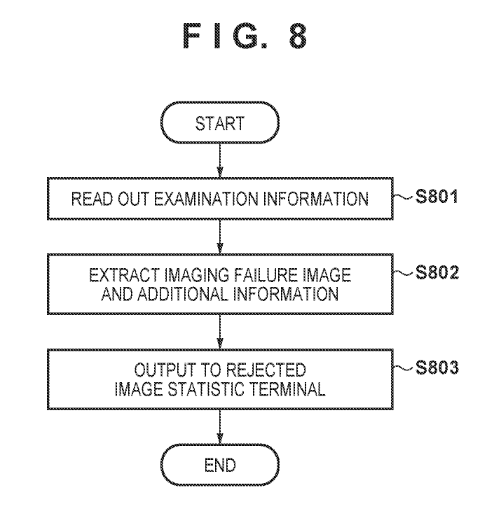

The procedure of processing executed by the rejected image statistic output unit 208 according to an embodiment of the present invention will be described next with reference to the flowchart of FIG. 8. An example of processing in which after the imaging success/failure identification unit 209 assigns the imaging success/failure item 607 to examination information, the rejected image statistic output unit 208 outputs rejected images and the additional information of the rejected images to the rejected image statistic terminal 40 will be described in detail. The processing of the rejected image statistic output unit 208 is executed at an arbitrary timing after completion of the processing of the imaging success/failure identification unit 209. For example, the processing is executed at the timing of an execution instruction via the operation unit 104 or executed on the background by designating a desired execution date/time.

In step S801, the rejected image statistic output unit 208 reads out examination information to be output to the rejected image statistic terminal 40 from the examination information storage unit 203. In step S802, the rejected image statistic output unit 208 extracts, from the examination information read out in step S801, captured images to which imaging failure information is assigned by the imaging success/failure identification unit 209 and the additional information of the captured images. In the example of examination information of the table 60 shown in FIG. 6, images IM001, IM002, IM003, and IM007, and the additional information of these images are extracted. In step S803, the rejected image statistic output unit 208 outputs the captured images and the additional information extracted in step S802 to the rejected image statistic terminal 40. The series of processes of FIG. 8 thus ends.

As described above, according to this embodiment, it is possible to assign information representing an imaging success/failure state to each captured image independently of information representing a rejected image state. It is also possible to output only captured images with assigned imaging failure information to the rejected image statistic terminal 40.

For example, for images obtained by a tomosynthesis imaging protocol, the imaging failure information can be assigned to all images changed to the rejected image state with the intention of an imaging technique failure, like images IM001, IM002, and IM003 of the examination information in the table 60. Hence, the images can be output to the rejected image statistic terminal 40 as the rejected image analysis target.

In addition, for a tomosynthesis imaging protocol including an effective image, like imaging protocol P003 of the examination information in the table 60, image IM005 changed to the rejected image state with the intention of non-transfer is regarded as an imaging success. For this reason, the image is not a rejected image analysis target and is not output to the rejected image statistic terminal 40.

On the other hand, for imaging protocols other than the tomosynthesis imaging protocol, the rejected image state is set with the intention of an imaging technique failure, and the imaging failure information can be assigned to rejected images. Hence, the images can be output as the rejected image analysis target to the rejected image statistic terminal 40.

As described above, only images necessary for rejected image analysis are output to the rejected image statistic terminal 40. This allows the side of the rejected image statistic terminal 40 to save time to sort necessary images and unnecessary images for rejected image analysis and improve the rejected image analysis efficiency. The improvement of the rejected image analysis efficiency can contribute to improvement of the skill of the imaging technician. Along with the improvement of the skill, ineffective exposure can be reduced, contributing to protection of a patient.

### Second Embodiment

<Outline>

The second embodiment is different from the first embodiment in that an image captured by a DX imaging protocol is used as a captured image, and a replicated image is used as a processed image based on the captured image. More specifically, if an image (still image) captured by a DX imaging protocol and replicated images generated from the captured image include an effective image (an image that is not rejected image-instructed), information representing an imaging success is assigned to all images. On the other hand, if no effective image is included, information representing an imaging failure is assigned to all images. Then, each image with the assigned information representing an imaging failure and the additional information of the image are output to a rejected image statistic terminal. The second embodiment will be described below in detail.

<Arrangements>

The arrangements of a control system and an X-ray imaging system according to this embodiment are the same as in the first embodiment, and a detailed description thereof will be omitted.

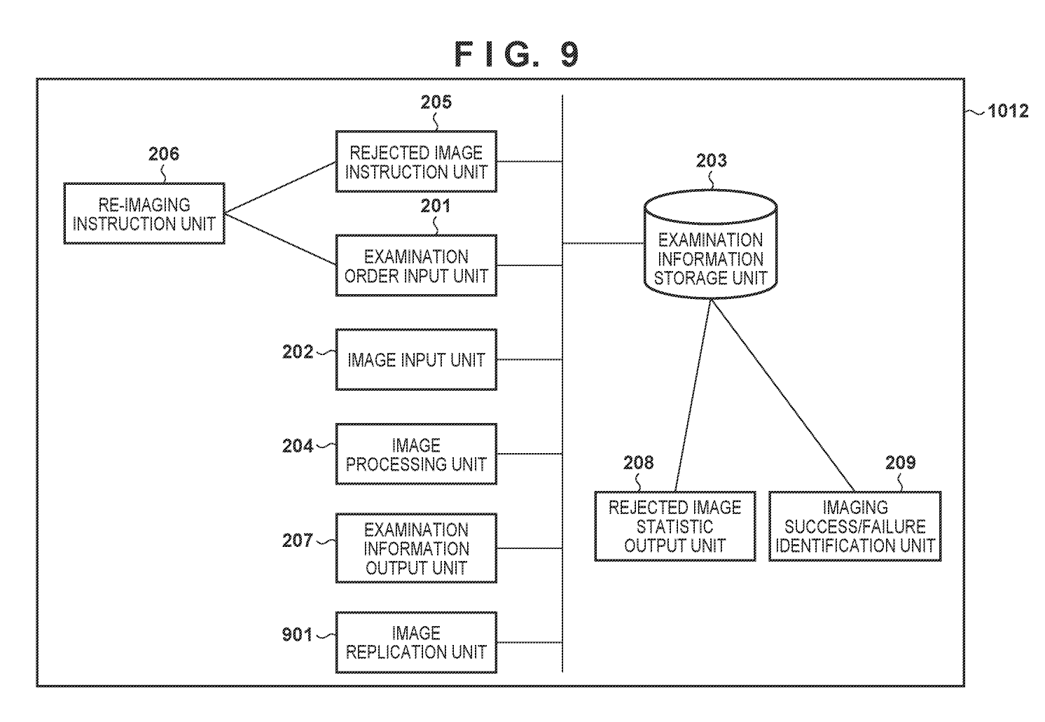

FIG. 9 is a block diagram showing an example of the arrangement of an X-ray imaging control unit according to an embodiment of the present invention. An X-ray imaging control unit 1012 according to this embodiment includes an image replication unit 901 in addition to the arrangement described with reference to FIG. 2. The image replication unit 901 replicates a designated captured image and the additional information of the captured image and stores the replication contents in an examination information storage unit 203.

<Example of Imaging Screen>

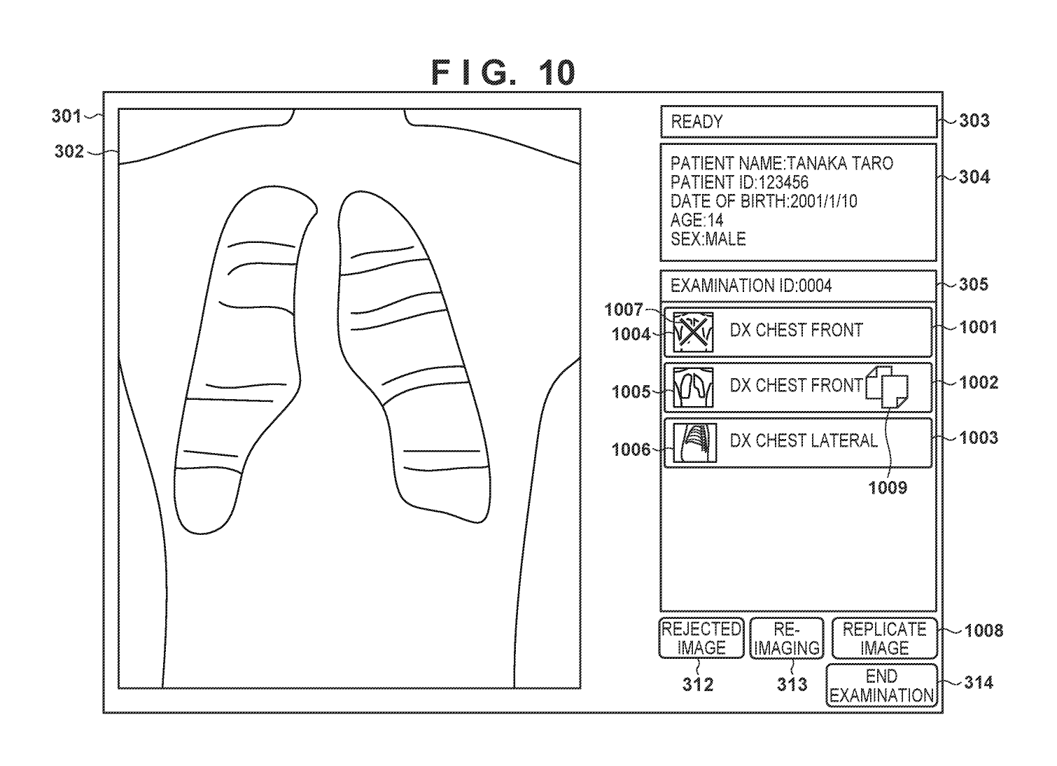

FIG. 10 shows an example of an imaging screen according to an embodiment of the present invention. The same reference numerals as in FIG. 3 described in the first embodiment denote the same constituent elements in FIG. 10. An examination order display region 305 displays imaging protocols 1001, 1002, and 1003 included in an examination. The name of an imaging technique is displayed in correspondence with each imaging protocol. The imaging protocols are provided with thumbnail display regions 1004, 1005, and 1006 that display the thumbnails of images captured and generated by the imaging protocols. A symbol 1007 representing a rejected image is displayed on the thumbnail of a rejected image. An image replication button 1008 is a button used to instruct image replication. A symbol 1009 representing replication is displayed on the imaging protocol of a replicated image.

<Processing>

The procedure of a series of processes from the start to the end of an examination according to the second embodiment is the same as the processing shown in FIG. 4 described in the first embodiment except some differences. The different points will mainly be described below.

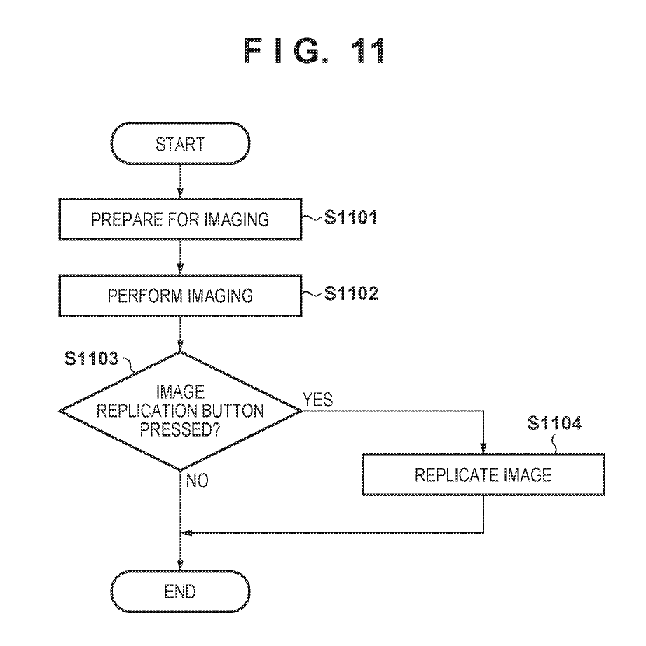

Details of the processing of step S403 according to an embodiment of the present invention will be described below with reference to the flowchart of FIG. 11.

The processing of step S1101 is the same as the processing of step S501 in FIG. 5, and a description thereof will be omitted. In step S1102, a control apparatus 101 executes imaging using an imaging protocol. More specifically, an X-ray tube 1021 irradiates an object with X-rays, and a sensor 1022 detects the X-rays transmitted through the object. An image input unit 202 acquires a captured image obtained as a result, and an image processing unit 204 performs image processing such as correction processing, tone processing, or frequency processing for the acquired captured image. The captured image is associated with examination information and stored in the examination information storage unit 203. At this time, an imaging screen 301 displays the captured image and additional image information. The captured image is displayed in a preview image display region 302, and the thumbnail of the captured image is displayed in the thumbnail display regions 1004, 1005, and 1006 on the imaging protocol used for the imaging.

In step S1103, the control apparatus 101 determines whether the image replication button 1008 is pressed via an operation on an operation unit 104. If the image replication button 1008 is pressed (YES in step S1103), the process advances to step S1104. On the other hand, if the image replication button 1008 is not pressed (NO in step S1103), the processing ends. In step S1104, the control apparatus 101 causes the image replication unit 901 to replicate a designated captured image and the additional information of the image, and stores the replication contents in the examination information storage unit 203. The series of processes in FIG. 11 thus ends.

<Example of Examination Information>

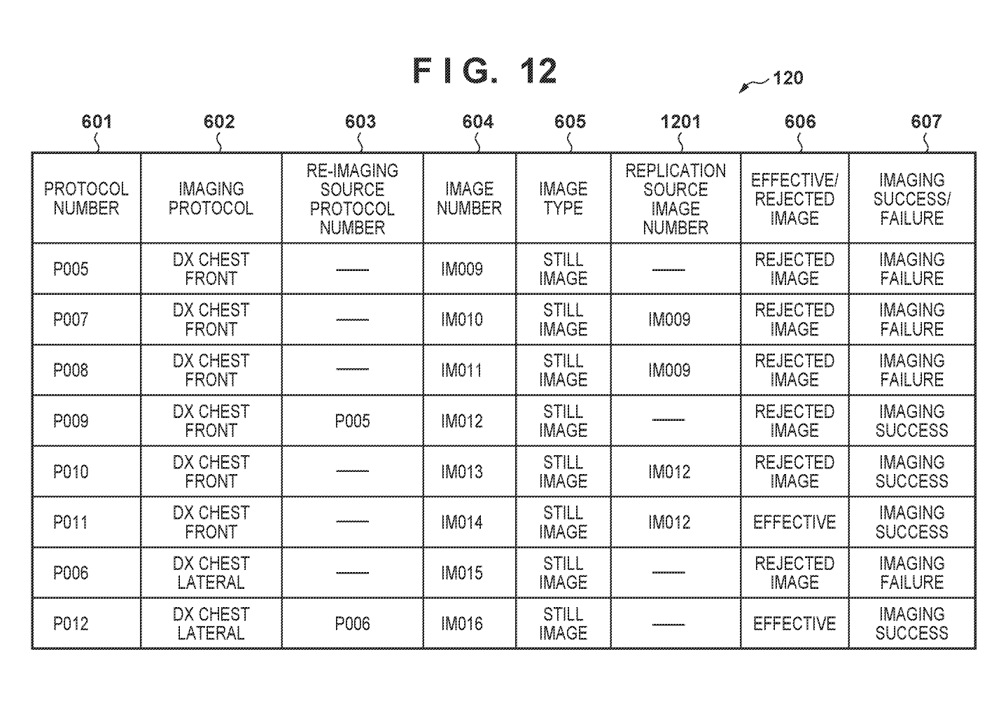

An example of examination information according to an embodiment of the present invention will be described next with reference to FIG. 12. A table 120 shows some pieces of examination information after the end of an examination, which are stored in the examination information storage unit 203, in a table format.

A replication source image number item 1201 is the number of an image number item 604 of a replication source, which is assigned to an image replicated by the processing of the image replication unit 901. The remaining items are the same as those of the table 60 shown in FIG. 6 described in the first embodiment, and a description thereof will be omitted.

<Acquisition Processing of Items (Other than Imaging Success/Failure Item 607) of Table 120>

What kind of processing is performed up to the end of an examination to obtain the pieces of examination information other than an imaging success/failure item 607 of the table 120 shown in FIG. 12 will be described below with reference to FIGS. 4 and 5 again.

First, imaging protocols P005 and P006 are input in step S401 of FIG. 4, and the examination is started in step S402. In the imaging processing and post-processing of step S403, first, imaging is performed based on imaging protocol P005 in steps S1101 and S1102 of FIG. 11. Projected image IM009 is thus acquired.

To perform a plurality of different image processes and compare images, the image replication button 1008 is pressed in step S1103. As a result, replicated images IM010 and IM011 of captured image IM009 are acquired in step S1104. Note that there is a mechanism for associating imaging protocol P005 with a captured image in a one-to-one correspondence. For this reason, along with the image replication, imaging protocols P007 and P008 to be associated with the replicated images IM010 and IM011, respectively, are replicated based on imaging protocol P005. Images IM009, IM010, and IM011 undergo image processing using different image processing parameters.

Assume that the operator compares all images IM009, IM010, and IM011, understands that the imaging technique for captured image IM009 has failed, and presses a re-imaging button 313 in step S404 of FIG. 4. By the subsequent processing of step S405, first, images IM009, IM010, and IM011 are changed to the rejected image state. Imaging protocol P009 that is the same as imaging protocol P005 is added to the examination order.

Next, the process returns to step S403. To further perform imaging based on re-imaging protocol P009, the process advances to step S1102, and captured image IM012 is acquired. In step S1104, replicated images IM013 and IM014 of captured image IM012 are acquired. Note that along with the image replication, imaging protocols P010 and P011 to be associated with the replicated images IM013 and IM014, respectively, are replicated based on imaging protocol P009. Images IM012, IM013, and IM014 undergo image processing using different image processing parameters.

Assume that the operator compares images IM012, IM013, and IM014, and determines that image IM014 is most appropriate for a diagnosis. Here, since effective image IM014 is included, the re-imaging button 313 is not pressed in step S404, and the process advances to step S406. In step S406, to prevent images IM012 and IM013 inappropriate for a diagnosis from being transferred to a PACS terminal 30, a rejected image button 312 is pressed. Next, in step S407, images IM012 and IM013 are changed to the rejected image state. Since unexecuted imaging protocol P006 remains, an examination end button 314 is not pressed in step S408, and the process returns to step S403.

Next, captured image IM015 is acquired by the imaging in step S1102. If the operator determines that captured image IM015 need not be compared based on a plurality of image processes, the image replication button 1008 is not pressed in step S1103. Here, assume that the operator observes captured image IM015, understands that the imaging technique has failed, and presses the re-imaging button 313 in step S404. Next, image IM015 obtained by imaging protocol P006 is changed to the rejected image state by the processing of step S405. Imaging protocol P012 that is the same as imaging protocol P006 is added to the examination order.

Next, the process returns to step S403. To further perform imaging using re-imaging protocol P012, the process advances to step S1102, and captured image IM016 is acquired. The operator is assumed to determine that captured image IM016 is appropriate for a diagnosis. In this case, the re-imaging button 313 is not pressed in step S404, the rejected image button 312 is not pressed in step S406, and the process advances to step S408. Since the imaging processes of all imaging protocols are completed, the examination end button 314 is pressed in step S408, and the examination ends.

By the series of processes described above, the items of the table 120 except the imaging success/failure item in FIG. 12 are obtained.

<Acquisition Processing of Imaging Success/Failure Item 607 of Table 120>

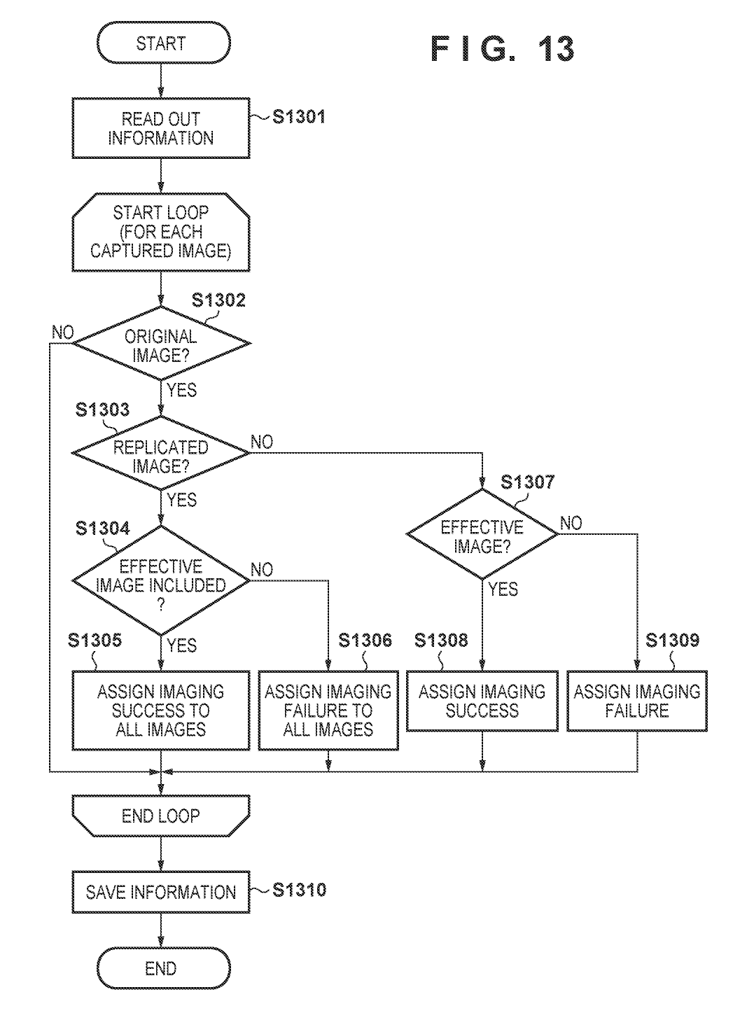

The procedure of processing executed by an imaging success/failure identification unit 209 for examination information after the end of the examination according to an embodiment of the present invention will be described next with reference to the flowchart of FIG. 13. What kind of processing is performed to obtain the imaging success/failure item 607 of the table 120 shown in FIG. 12 will be described here.

In step S1301, the imaging success/failure identification unit 209 reads out examination information to be processed from the examination information storage unit 203. Here, the examination information of the table 120 shown in FIG. 12 is read out. Next, the processes of steps S1302 to S1309 are performed as a loop for the images in the examination information sequentially from the top. After the end of the loop, the process advances to step S1310. First, processing is performed for image IM009 at the top in the table 120.

In step S1302, the imaging success/failure identification unit 209 determines whether the image is an original image or a replicated image. If the image is an original image (YES in step S1302), the process advances to step S1303. On the other hand, if the image is a replicated image, the process returns to the start of the loop, and processing is performed for the next image. Here, since image IM009 is an original image, the process advances to step S1303.

In step S1303, the imaging success/failure identification unit 209 determines whether a replicated image of the image exists. If a replicated image exists (YES in step S1303), the process advances to step S1304. On the other hand, if no replicated image exists (NO in step S1303), the process advances to step S1307. Here, since image IM009 has replicated images IM010 and IM011, the process advances to step S1304.

In step S1304, the imaging success/failure identification unit 209 determines whether an effective image is included in the image and all replicated images of the image. If an effective image is included (YES in step S1304), the process advances to step S1305. On the other hand, if no effective image is included (NO in step S1304), the process advances to step S1306. Here, since all of original image IM009 and replicated images IM010 and IM011 are in the rejected image state, the process advances to step S1306.

In step S1306, the imaging success/failure identification unit 209 assigns imaging failure information to all of the image and the replicated images of the image. Here, imaging failure information is assigned to images IM009, IM010, and IM011. After that, the process returns to step S1302 at the start of the loop.

As the result of determination of step S1302, processing for replicated images IM010 and IM011 is not performed (NO in step S1302), and the loop advances to next original image IM012. Since image IM012 is an original image (YES in step S1302), the process advances to step S1303. Here, since image IM012 has replicated images IM013 and IM014 (YES in step S1303), the process advances to step S1304. Since replicated image IM014 is an effective image among original image IM012 and replicated images IM013 and IM014 (YES in step S1304), the process advances to step S1305.

In step S1305, the imaging success/failure identification unit 209 assigns imaging success information to all of the image and the replicated images of the image. Here, imaging success information is assigned to images IM012, IM013, and IM014. After that, the process returns to step S1302 at the start of the loop.

As the result of determination of step S1302, processing for replicated images IM013 and IM014 is not performed (NO in step S1302), and the loop advances to next original image IM015. Since image IM015 is an original image (YES in step S1302), the process advances to step S1303. Since image IM015 has no replicated image (NO in step S1303), the process advances to step S1307.

In step S1307, the imaging success/failure identification unit 209 determines whether the image is an effective image. If the image is an effective image (YES in step S1307), the process advances to step S1308. On the other hand, if the image is not an effective image (NO in step S1307), the process advances to step S1309. Here, since image IM015 is a rejected image (NO in step S1307), the process advances to step S1309.

In step S1309, the imaging success/failure identification unit 209 assigns imaging failure information to the image. Here, imaging failure information is assigned to image IM015. After that, the process returns to step S1302 at the start of the loop, and processing is performed for image IM016 next. Since image IM016 is an original image (YES in step S1302), the process advances to step S1303. Since image IM016 has no replicated image (NO in step S1303), the process advances to step S1307. Since image IM016 is an effective image (YES in step S1307), the process advances to step S1308.

In step S1308, the imaging success/failure identification unit 209 assigns imaging success information to the image. Here, imaging success information is assigned to image IM016. Since the loop processing is thus completed for all images, the process advances to step S1310. In step S1310, the imaging success/failure identification unit 209 stores the examination information changed by the series of processes of steps S1302 to S1309 in the examination information storage unit 203. By the processes described above, the information of the imaging success/failure item 607 of the table 120 is obtained.

<Processing of Rejected Image Statistic Output Unit 208>

The processing of a rejected image statistic output unit 208 according to this embodiment is basically the same as the processing shown in FIG. 8 of the first embodiment. In the example of the table 120 shown in FIG. 12, however, the second embodiment is different in that images IM009, IM010, IM011, and IM015 are extracted in step S802.

As described above, according to this embodiment, it is possible to assign information representing an imaging success/failure state to each captured image independently of information representing a rejected image state. It is also possible to output only captured images with assigned imaging failure information to a rejected image statistic terminal 40.

For example, for captured images having replicated images, the imaging failure information can be assigned to all images changed to the rejected image state with the intention of an imaging technique failure, like images IM009, IM010, and IM011 of the examination information in the table 120. Hence, the images can be output to the rejected image statistic terminal 40 as the rejected image analysis target.

In addition, for captured images having replicated images in the examination information of the table 120, each of images IM012 and IM013 changed to the rejected image state with the intention of non-transfer is regarded as an imaging success. For this reason, the images are not rejected image analysis targets and are not output to the rejected image statistic terminal 40.

On the other hand, for captured images having no replicated image, the rejected image state is set with the intention of an imaging technique failure, and the imaging failure information can be assigned to rejected images. Hence, the images can be output as the rejected image analysis target to the rejected image statistic terminal 40.

As described above, only images necessary for rejected image analysis are output to the rejected image statistic terminal 40. This allows the side of the rejected image statistic terminal 40 to save time to sort necessary images and unnecessary images for rejected image analysis and improve the rejected image analysis efficiency.

### Third Embodiment

<Outline>

In the first embodiment, the target to be output to the rejected image statistic terminal includes an image with assigned information representing an imaging failure and the additional information of the image. The third embodiment is different from that first embodiment in that the target to be output to the rejected image statistic terminal includes a rejected image with assigned imaging success/failure information and the additional information of the rejected image.

<Arrangement>

The arrangements of a control system and an X-ray imaging system according to this embodiment are the same as in the first embodiment, and a detailed description thereof will be omitted. In addition, the arrangement of an X-ray imaging control unit 1012 according to this embodiment is the same as that described with reference to FIGS. 1 and 2 in the first embodiment. An example of an imaging screen according to this embodiment is the same as in FIG. 3 of the first embodiment. A series of processes from the start to the end of an examination according to this embodiment is also the same as the processes shown in FIGS. 4 and 5. Processing for obtaining examination information is also the same as the processes shown in FIGS. 6 and 7.

<Processing of Rejected Image Statistic Output Unit 208>

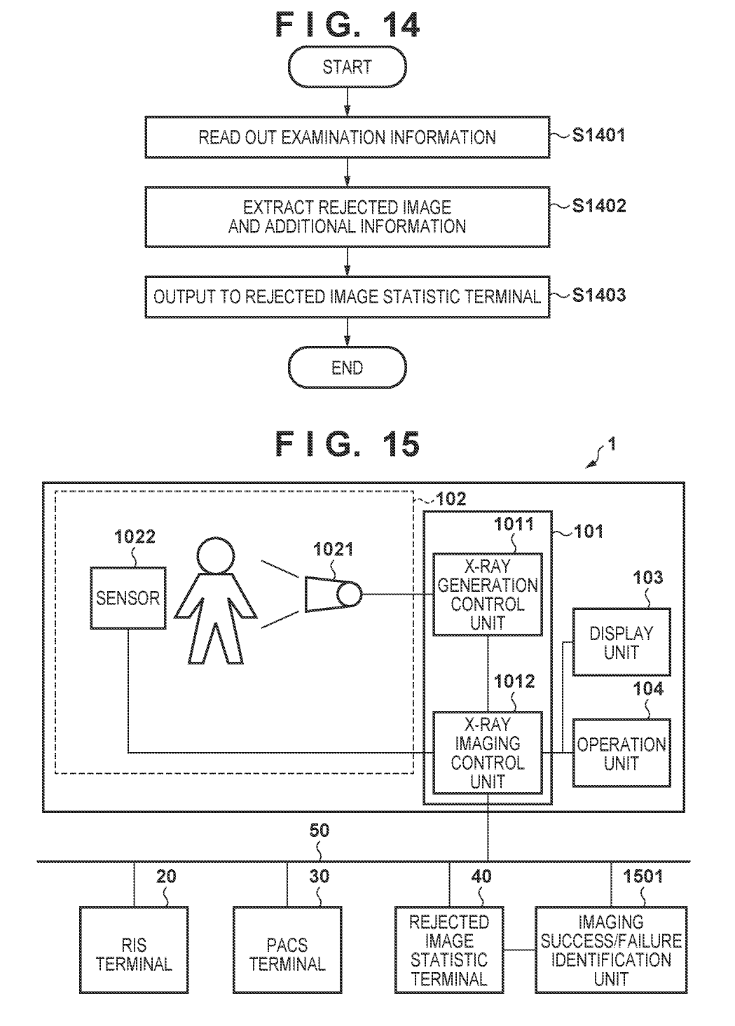

The procedure of processing executed by a rejected image statistic output unit 208 according to an embodiment of the present invention will be described next with reference to the flowchart of FIG. 14. An example of processing in which after an imaging success/failure identification unit 209 assigns imaging success/failure information to examination information, the rejected image statistic output unit 208 outputs rejected images and the additional information of the rejected images to a rejected image statistic terminal 40 will be described in detail.

The processing of step S1401 is the same as the processing of step S801 of FIG. 8. In step S1402, the rejected image statistic output unit 208 extracts images in the rejected image state and the additional information of the images from the examination information read out in step S1401. In the example of examination information of a table 60 shown in FIG. 6, images IM001, IM002, IM003, IM005, and IM007 are extracted. The processing of step S1403 is the same as the processing of step S803 of FIG. 8.

As described above, according to this embodiment, it is possible to assign information representing an imaging success/failure state to each captured image independently of information representing a rejected image state.

For example, for images obtained by a tomosynthesis imaging protocol, the imaging failure information can be assigned to all images changed to the rejected image state with the intention of an imaging technique failure, like images IM001, IM002, and IM003 of the examination information in the table 60. In addition, for a tomosynthesis imaging protocol including an effective image, like imaging protocol P003 of the examination information in the table 60, image IM005 changed to the rejected image state with the intention of non-transfer represents an imaging success.

On the other hand, for imaging protocols other than the tomosynthesis imaging protocol, the rejected image state is set with the intention of an imaging technique failure, and the imaging failure information can be assigned to rejected images. In this way, all rejected images to which information representing an imaging success/failure state is assigned are output to the rejected image statistic terminal 40. However, the side of the rejected image statistic terminal 40 need only perform filtering to refer to only imaging failure images at the time of rejected image analysis. This allows the side of the rejected image statistic terminal 40 to save time to sort necessary images and unnecessary images for rejected image analysis and improve the rejected image analysis efficiency.

### Fourth Embodiment

<Outline>

In the second embodiment, the target to be output to the rejected image statistic terminal includes an image with assigned information representing an imaging failure and the additional information of the image. The fourth embodiment is different from that second embodiment in that the target to be output to the rejected image statistic terminal includes a rejected image with assigned imaging success/failure information and the additional information of the rejected image.

<Arrangement>

The arrangements of a control system and an X-ray imaging system according to this embodiment are the same as in the first embodiment, and a detailed description thereof will be omitted. In addition, the arrangement of an X-ray imaging control unit 1012 according to this embodiment is the same as that described with reference to FIG. 9 in the second embodiment. An example of an imaging screen according to this embodiment is the same as in FIG. 10 of the second embodiment. A series of processes from the start to the end of an examination according to this embodiment is also the same as the processes shown in FIGS. 4 and 11. Processing for obtaining examination information is also the same as the processes shown in FIGS. 12 and 13 of the second embodiment.

<Processing of Rejected Image Statistic Output Unit 208>

The procedure of processing executed by a rejected image statistic output unit 208 according to an embodiment of the present invention will be described next with reference to the flowchart of FIG. 14. The processing of step S1401 is the same as the processing of step S801 of FIG. 8. In step S1402, the rejected image statistic output unit 208 extracts images in the rejected image state and the additional information of the images from the examination information read out in step S1401. In the example of examination information of a table 120 shown in FIG. 12, images IM009, IM010, IM011, IM012, IM013, and IM015 are extracted. The processing of step S1403 is the same as the processing of step S803 of FIG. 8.

As described above, according to the fourth embodiment, it is possible to assign information representing an imaging success/failure state to each captured image independently of information representing a rejected image state.

For example, for captured images having replicated images, the imaging failure information can be assigned to all images changed to the rejected image state with the intention of an imaging technique failure, like images IM009, IM010, and IM011 in the table 120. In addition, for captured images having replicated images in the table 120, each of images IM012 and IM013 changed to the rejected image state with the intention of non-transfer is regarded as an imaging success.

On the other hand, for captured images having no replicated image, the rejected image state is set with the intention of an imaging technique failure, and the imaging failure information can be assigned to rejected images. In this way, all rejected images to which information representing an imaging success/failure state is assigned are output to a rejected image statistic terminal 40. However, the side of the rejected image statistic terminal 40 need only perform filtering to refer to only imaging failure images at the time of rejected image analysis. This allows the side of the rejected image statistic terminal 40 to save time to sort necessary images and unnecessary images for rejected image analysis and improve the rejected image analysis efficiency.

### Fifth Embodiment

<Outline>

In the fifth embodiment, an example will be described in which an imaging success/failure identification unit is provided outside a control apparatus, and information representing an imaging success/failure is assigned to an image stored in a rejected image statistic terminal, unlike the first embodiment.

<Arrangements>

FIG. 15 is a block diagram showing an example of the arrangement of a control system according to the fifth embodiment. The same reference numerals as in FIG. 1 described in the first embodiment denote the same constituent elements in FIG. 15. Reference numeral 1501 denotes an imaging success/failure identification unit. The imaging success/failure identification unit 1501 identifies, based on the additional information of a rejected image stored in a rejected image statistic terminal 40, whether the rejected image is generated by an imaging failure.

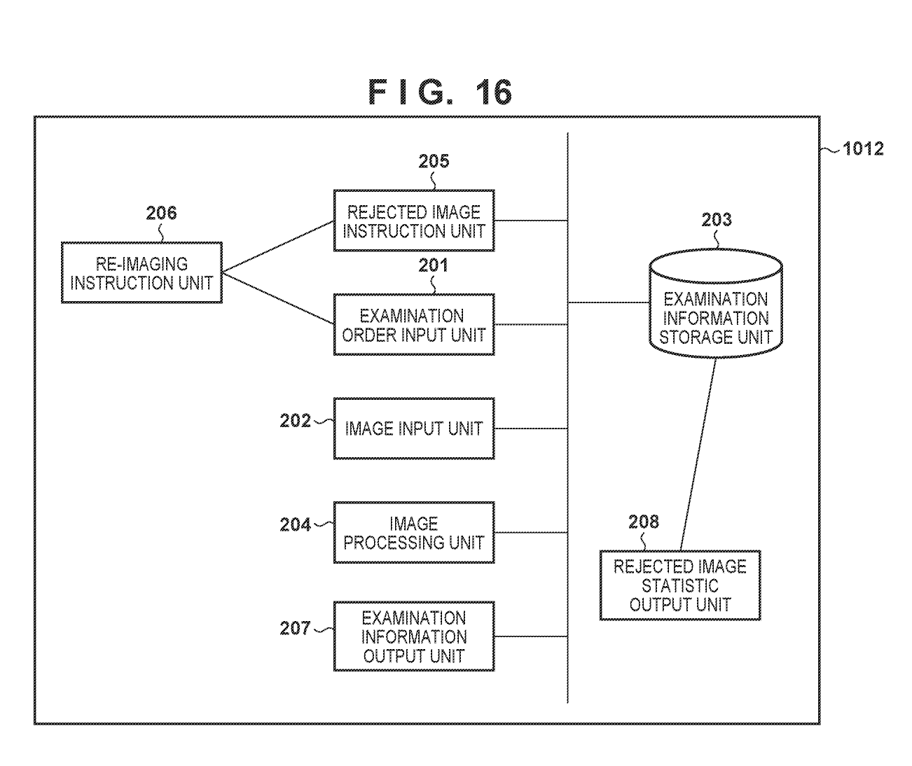

FIG. 16 shows an example of a functional arrangement configured to implement the fifth embodiment in an X-ray imaging control unit 1012 shown in FIG. 15. Note that the same reference numerals as in FIG. 2 described in the first embodiment denote the same constituent elements in FIG. 16. The X-ray imaging control unit 1012 according to this embodiment is different from that in FIG. 2 in that an imaging success/failure identification unit 209 is not included.

An example of an imaging screen according to the fifth embodiment is the same as the first embodiment, as shown in FIG. 3. A series of procedures from the start to the end of an examination according to the fifth embodiment is the same as in FIGS. 4 and 5 described in the first embodiment. An example of processing for obtaining examination information necessary for an explanation of the function of the fifth embodiment is the same as in FIG. 6 described in the first embodiment.

An example of processing of casing a rejected image statistic output unit 208 to output a rejected image and the additional information of the rejected image to the rejected image statistic terminal 40 after the end of an examination in the fifth embodiment is the same as in FIG. 14 described in the third embodiment. Note that the processing of the rejected image statistic output unit 208 is executed at an arbitrary timing after the end of the examination. For example, the processing is executed by instructing execution via an operation unit 104 or executed on the background by designating a desired execution date/time.

The procedure of processing of the imaging success/failure identification unit 1501 after information output from the rejected image statistic output unit 208 is input to the rejected image statistic terminal 40 in the fifth embodiment is the same as in FIG. 7 described in the first embodiment, and only some different points will be described. Note that the processing of the imaging success/failure identification unit 1501 is executed at an arbitrary timing after information output from the rejected image statistic output unit 208 is input to the rejected image statistic terminal 40 until the rejected image statistic terminal 40 performs rejected image analysis. For example, the processing is executed immediately after reception of an input from the rejected image statistic output unit 208, at the timing of an execution instruction via an operation unit (not shown) of the rejected image statistic terminal 40, or at the timing of rejected image analysis by the rejected image statistic terminal 40.

In step S701 according to the fifth embodiment, a rejected image associated with examination information as a processing target and additional information are read out from the rejected image statistic terminal 40. Here, a rejected image associated with examination information and additional information are read out in accordance with a table 60 shown in FIG. 6.

In step S709 according to the fifth embodiment, information changed by a series of processes of steps S702 to S708 is stored in the rejected image statistic terminal 40. By the processes described above, the imaging success/failure item of the table 60 is obtained.

As described above, according to this embodiment, it is possible to assign information representing an imaging success/failure state to each rejected image stored in the rejected image statistic terminal 40 independently of information representing a rejected image state.

For example, for images obtained by a tomosynthesis imaging protocol, the imaging failure information can be assigned to all images changed to the rejected image state with the intention of an imaging technique failure, like images IM001, IM002, and IM003 of the examination information in the table 60. In addition, for a tomosynthesis imaging protocol including an effective image, like imaging protocol P003 of the examination information in the table 60, image IM005 changed to the rejected image state with the intention of non-transfer is regarded as an imaging success.

On the other hand, for imaging protocols other than the tomosynthesis imaging protocol, the rejected image state is set with the intention of an imaging technique failure, and the imaging failure information can be assigned to rejected images. Although all rejected images are output from the rejected image statistic output unit 208 to the rejected image statistic terminal 40, the side of the rejected image statistic terminal 40 need only assign the information representing the imaging success/failure state and perform filtering to refer to only imaging failure images at the time of rejected image analysis. This allows the side of the rejected image statistic terminal 40 to save time to sort necessary images and unnecessary images for rejected image analysis and improve the rejected image analysis efficiency.

### Sixth Embodiment

<Outline>

In the sixth embodiment, an example will be described in which an imaging success/failure identification unit is provided outside a control apparatus, and information representing an imaging success/failure is assigned to an image stored in a rejected image statistic terminal, unlike the second embodiment.

<Arrangements>

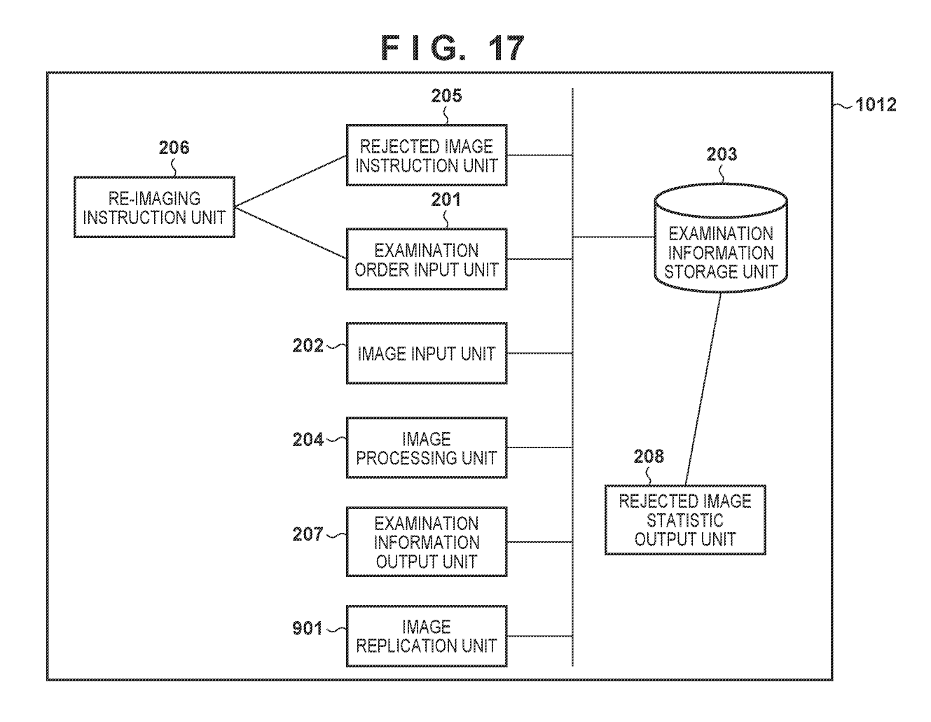

The arrangement of a control system according to the sixth embodiment is the same as in FIG. 15 described in the fifth embodiment. FIG. 17 shows an example of a functional arrangement configured to implement the sixth embodiment in an X-ray imaging control unit 1012 shown in FIG. 15. The same reference numerals as in FIG. 9 described in the second embodiment denote the same constituent elements in FIG. 17. The X-ray imaging control unit 1012 according to this embodiment is different from that in FIG. 9 in that an imaging success/failure identification unit 209 is not included.

An example of an imaging screen according to the sixth embodiment is the same as in FIG. 10 described in the second embodiment. A series of procedures from the start to the end of an examination according to the sixth embodiment is the same as in FIGS. 4 and 11 described in the second embodiment. An example of processing for obtaining examination information necessary for an explanation of the function of the sixth embodiment is the same as in FIG. 12 described in the second embodiment.

An example of processing of casing a rejected image statistic output unit 208 to output a rejected image and the additional information of the rejected image to the rejected image statistic terminal 40 after the end of an examination in the sixth embodiment is the same as in FIG. 14 described in the third embodiment. Note that the processing of the rejected image statistic output unit 208 is executed at an arbitrary timing after the end of the examination. For example, the processing is executed by instructing execution via an operation unit 104 or executed on the background by designating a desired execution date/time.

The procedure of processing of an imaging success/failure identification unit 1501 after information output from the rejected image statistic output unit 208 is input to the rejected image statistic terminal 40 in the sixth embodiment is the same as in FIG. 13 described in the second embodiment, and only some different points will be described. Note that the processing of the imaging success/failure identification unit 1501 is executed at an arbitrary timing after information output from the rejected image statistic output unit 208 is input to the rejected image statistic terminal 40 until the rejected image statistic terminal 40 performs rejected image analysis. For example, the processing is executed immediately after reception of an input from the rejected image statistic output unit 208, at the timing of an execution instruction via an operation unit (not shown) of the rejected image statistic terminal 40, or at the timing of rejected image analysis by the rejected image statistic terminal 40.

In step S1301 according to the sixth embodiment, a rejected image associated with examination information as a processing target and additional information are read out from the rejected image statistic terminal 40. Here, a rejected image associated with examination information and additional information are read out in accordance with a table 120 shown in FIG. 12.

In step S1310 according to the sixth embodiment, information changed by a series of processes of steps S1302 to S1309 is stored in the rejected image statistic terminal 40. By the processes described above, the imaging success/failure item of the table 120 is obtained.

As described above, according to this embodiment, it is possible to assign information representing an imaging success/failure state to each rejected image stored in the rejected image statistic terminal 40 independently of information representing a rejected image state.

For example, for captured images having replicated images, the imaging failure information can be assigned to all images changed to the rejected image state with the intention of an imaging technique failure, like images IM009, IM010, and IM011 in the table 120. In addition, for captured images having replicated images in the table 120, each of images IM012 and IM013 changed to the rejected image state with the intention of non-transfer is regarded as an imaging success.

On the other hand, for captured images having no replicated image, the rejected image state is set with the intention of an imaging technique failure, and the imaging failure information can be assigned to rejected images. Although all rejected images are output from the rejected image statistic output unit 208 to the rejected image statistic terminal 40, the side of the rejected image statistic terminal 40 need only assign the information representing the imaging success/failure state and perform filtering to refer to only imaging failure images at the time of rejected image analysis. This allows the side of the rejected image statistic terminal 40 to save time to sort necessary images and unnecessary images for rejected image analysis and improve the rejected image analysis efficiency.

According to the present invention, it is possible to save time to select images necessary for rejected image analysis and improve the rejected image analysis efficiency.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-159680, filed Aug. 16, 2016, which is hereby incorporated by reference wherein in its entirety.

What is claimed is:

1. A control apparatus comprising:
an instruction unit configured to receive an instruction to change one of a captured image and an image that is a processed image based on the captured image to a rejected image; and
an assigning unit configured to assign information representing an imaging success/failure to the image based on information of an imaging protocol of the image and information representing whether the image is the rejected image instructed by the instruction unit.

2. The apparatus according to claim 1, wherein the captured image is a projected image obtained by tomosynthesis imaging, and
the processed image is a reconstructed image obtained from the projected image.

3. The apparatus according to claim 2, wherein if the imaging protocol is a tomosynthesis imaging protocol, and an effective image that is not instructed by the instruction unit is included in the projected image and the reconstructed image, the assigning unit assigns information representing an imaging success to all of the projected image and the reconstructed image.

4. The apparatus according to claim 2, wherein if the imaging protocol is a tomosynthesis imaging protocol, and an effective image that is not instructed by the instruction unit is not included in the projected image and the reconstructed image, the assigning unit assigns information representing an imaging failure to all of the projected image and the reconstructed image.

5. The apparatus according to claim 1, wherein if the imaging protocol is not a tomosynthesis imaging protocol, and the image is an effective image that is not instructed by the instruction unit, the assigning unit assigns information representing an imaging success to the image.

6. The apparatus according to claim 1, wherein if the imaging protocol is not a tomosynthesis imaging protocol, and the image is the rejected image instructed by the instruction unit, the assigning unit assigns information representing an imaging failure to the image.

7. The apparatus according to claim 1, wherein the processed image is a replicated image obtained from the captured image, and
the imaging protocol is a DX imaging protocol.

8. The apparatus according to claim 7, wherein if the image is an original image that is not the replicated image, a replicated image of the image exists, and an effective image that is not instructed by the instruction unit is included in the original image and the replicated image, the assigning unit assigns information representing an imaging success to all of the original image and the replicated image.

9. The apparatus according to claim 7, wherein if the image is an original image that is not the replicated image, a replicated image of the image exists, and an effective image that is not instructed by the instruction unit is not included in the original image and the replicated image, the assigning unit assigns information representing an imaging failure to all of the original image and the replicated image.

10. The apparatus according to claim 7, wherein if the image is an original image that is not the replicated image, a replicated image of the image does not exist, and the image is an effective image that is not instructed by the instruction unit, the assigning unit assigns information representing an imaging success to the image.

11. The apparatus according to claim 7, wherein if the image is an original image that is not the replicated image, a replicated image of the image does not exist, and the image is the rejected image instructed by the instruction unit, the assigning unit assigns information representing an imaging failure to the image.

12. The apparatus according to claim 1, further comprising an output unit configured to output the image to which information representing an imaging failure is assigned by the assigning unit and additional information of the image,
wherein the additional information includes at least one piece of information of a protocol number, an imaging protocol, a re-imaging source protocol number, an image number, an image type, information representing a rejected image state, and the information representing the imaging success/failure.

13. The apparatus according to claim 1, further comprising an output unit configured to output the rejected image to which the information representing the imaging success/failure is assigned by the assigning unit and additional information of the rejected image,
wherein the additional information includes at least one piece of information of a protocol number, an imaging protocol, a re-imaging source protocol number, an image number, an image type, information representing a rejected image state, and the information representing the imaging success/failure.

14. The apparatus according to claim 12, wherein the output unit performs output to a rejected image statistic terminal.

15. The apparatus according to claim 1, further comprising an output unit configured not to output, to a rejected image statistic terminal, an image to which information representing an imaging success is assigned by the assigning unit and which is changed to the rejected image by the instruction unit and output, to the rejected image statistic terminal, an image to which information representing an imaging failure is assigned by the assigning unit and which is changed to the rejected image by the instruction unit.

16. A control apparatus comprising:
an instruction unit configured to receive an instruction to change an image to a rejected image;
a determination unit configured to determine whether to output the rejected image to a rejected image statistic terminal; and
an output unit configured to output the rejected image determined, by the determination unit, to be output to the rejected image statistic terminal.

17. A control system comprising:
an instruction unit configured to receive an instruction to change one of a captured image and an image that is a processed image based on the captured image to a rejected image; and
an assigning unit configured to assign information representing an imaging success/failure to the image based on information of an imaging protocol of the image and information representing whether the image is the rejected image instructed by the instruction unit.

18. The system according to claim 17, wherein the assigning unit assigns the information representing the imaging success/failure to an image stored in a rejected image statistic terminal.

19. A processing method executed by a control apparatus, comprising:
receiving an instruction to change one of a captured image and an image that is a processed image based on the captured image to a rejected image; and
assigning information representing an imaging success/failure to the image based on information of an imaging protocol of the image and information representing whether the image is the rejected image instructed in the receiving the instruction.

20. A processing method executed by a control apparatus, comprising:
receiving an instruction to change an image to a rejected image;
determining whether to output the rejected image to a rejected image statistic terminal; and
outputting the rejected image determined, in the determining whether to output the rejected image, to be output to the rejected image statistic terminal.

21. A non-transitory computer-readable storage medium that stores a program for executing a processing method executed by a control apparatus, comprising:
receiving an instruction to change one of a captured image and an image that is a processed image based on the captured image to a rejected image; and
assigning information representing an imaging success/failure to the image based on information of an imaging protocol of the image and information representing whether the image is the rejected image instructed in the receiving the instruction.

22. A non-transitory computer-readable storage medium that stores a program for executing a processing method executed by a control apparatus, comprising:
receiving an instruction to change an image to a rejected image;
determining whether to output the rejected image to a rejected image statistic terminal; and
outputting the rejected image determined, in the determining whether to output the rejected image, to be output to the rejected image statistic terminal.
